(19) United States
(12) Patent Application Publication
(10) Pub. No.: US 2024/0158407 A1
(43) Pub. Date: May 16, 2024

(54) METHOD FOR PREPARATION OF AMIDINES

(71) Applicant: VERSALIS S.P.A., San Donato Milanese (MI) (IT)

(72) Inventors: Nicola VECCHINI, MANTOVA (IT); Federico MONDINI, MANTOVA (IT)

(21) Appl. No.: 18/549,406

(22) PCT Filed: Mar. 3, 2022
(86) PCT No.: PCT/IB2022/051869

§ 371 Date: Sep. 7, 2023

(30) Foreign Application Priority Data
Mar. 8, 2021 (IT) 102021000005321

(51) Int. Cl. C07D 487/04

(52) U.S. Cl. CPC C07D 487/04

(57) ABSTRACT

A method for the preparation of amidines or their derivatives, includes the following step
synthesis of nitrile lactams by reaction between a lactam and an α-β unsaturated nitrile;
synthesis of N-(aminoalkyl) lactams by reducing the nitrile lactams;
and synthesis of amidines by dehydrating the N-(aminoalkyl) lactams.

### CROSS-REFERENCE TO RELATED APPLICATIONS

This application is a 35 U.S.C. § 371 National Stage patent application of PCT/IB2022/051869, filed on 3 Mar. 2022, which claims the benefit of Italian patent application 102021000005321, filed on 8 Mar. 2021, the disclosures of which are incorporated herein by reference in their entirety.

### TECHNICAL FIELD

The present disclosure relates to a process for the preparation of amidines. More particularly, the present disclosure relates to a method for producing amidines such as, for example, 1,8-Diazabicyclo-[5.4.0]-undec-7-ene (henceforth referred to in the abbreviated form DBU), or derivatives thereof from lactams, such as ε-Caprolactam and α,β unsaturated nitriles, such as acrylonitrile.

### BACKGROUND

It is well known that DBU is a versatile molecule that lends itself to numerous applications; indeed, the chemical reactions in which it can take part are varied.

A recent article by Jacques Muzart, “DBU: A Reaction Product Component' Chemistry Select 2020, vol. 5, 11608-11620, gives a detailed summary of this, ranging from the formation of salts to addition on C—C double bonds and much more. For these aspects DBU is used in the catalysis of polyurethanes, in the pharmaceutical industry, in ionic liquids and in general in organic synthesis. Further details on the application of DBU are also described in “1,8-Diazabicyclo[S4.0]undec-7-ene (DBU): A Versatile reagent in Organic Synthesis” Bhaskara Nand et al. Current Organic Chemistry, 2015, 19, 790-812.

In the prior art, the industrial production of DBU takes place mainly through three reaction steps. In the first step ε-caprolactam is reacted with acrylonitrile to obtain N-(2-cyanoethyl)-ε-caprolactam. In the second step, N-(2-cyanoethyl)-ε-caprolactam is hydrogenated to the corresponding amine in the presence of anhydrous ammonia and Nickel Raney catalyst. In the third step, N-(3-aminopropyl)-ε-caprolactam is dehydrated by acid catalysis to produce DBU. The industrially most complex step in the synthesis is hydrogenation in the presence of ammonia. The catalyst normally used is Nickel-Raney, which in its activated form is pyrophoric. Anhydrous ammonia is also a toxic gas and requires specific precautions and authorisations for its storage, use and transport. Below are some relevant documents of the prior art.

Patent DE1545855 describes, in its German version and in its English version in the countries to which it extends, a process for obtaining amidines (limited to the third step of the industrial process described above) with the following structure:

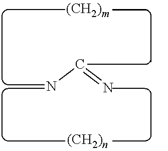

where m is an integer from 3 to 7, and n is an integer from 2 to 4, starting from N-(aminoalkyl) lactams of formula:

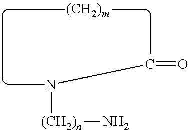

The process takes place through the dehydration of aminolactam catalysed by mineral or sulphonic acids (e.g. p-toluenesulphonic acid) in the presence of a solvent e.g. Xylene. The reaction mixture is heated to boiling point, the dehydration water formed is condensed with the solvent and then separated; the solvent is refluxed into the reaction flask. The patent does not describe the steps prior to dehydration, but makes reference to the prior art.

Patent EP0347757 A2 describes a method for the synthesis of cyanoalkyl lactams (the first step in the industrial process described above) through the reaction of a lactam and an α,β unsaturated nitrile using DBU itself as a basic catalyst; DBU can also be used as a solvent. The document does not mention the other reaction steps (second and third), but merely refers to the catalytic hydrogenation of cyanoalkyl lactam as in the prior art; indeed, in example 2, hydrogenation in the presence of Ni Raney and ammonia is described. In the patent, the use of DBU as a catalyst is justified as a substitute for KOH used in the first step of the industrial process, since, in order to be able to pass from the first step to the second, the base must first be neutralised, which is not necessary if DBU is used (example 3).

Patent CN101279973 B describes a method for the preparation of 1,8-Diazabicyclo-[5.4.0]-undec-7-ene, starting from ε-caprolactam and acrylonitrile, in the presence of ter-butyl or ter-amyl alcohol, as a solvent, and NaOH as a catalyst. The reaction product of this first step undergoes hydrogenation in the presence of anhydrous ammonia and Ni Raney as a catalyst. After hydrogenation, the mixture is neutralised with sulphuric acid, the solvent recovered and the reaction product is subjected to dehydration, with removal of water, as described in German patent DE1545855.

Patent publication CN109796458 A describes a method for the preparation of 1,8-diazabicyclo-[5.4.0]-undec-7-ene, again starting from ε-caprolactam and acrylonitrile. This time, the document no longer describes the hydrogenation step in the presence of ammonia, but introduces an alternative method using hydroquinone, anhydrous gaseous hydrochloric acid, dichloromethane, sodium perborate and ethylenediaminetetraacetic acid (EDTA). The process is considerably more complex than the others described, and while ammonia and Ni-Raney are eliminated, a highly aggressive agent (anhydrous HCl) is introduced as well as numerous chemical substances.

In patent publication JP2003286257, the first and third steps are carried out in the same way as described above (reaction of caprolactam with acrylonitrile by basic catalysis using KOH; dehydration by acid catalysis). The second step is conducted in the absence of ammonia and using Cobalt Raney as a catalyst. The result is 86% by weight of reduced product (primary amine of interest).

Patent EP0913388 B1 describes a method for obtaining amines by hydrogenation of nitriles without the use of ammonia. The novelty lies in the treatment to which the catalyst is subjected. The catalyst (Cobalt Raney or sponge catalyst) is treated with an aqueous lithium hydroxide solution or, alternatively, the reaction is carried out in the presence of this solution. Through this treatment, the catalyst must incorporate 0.1 to 100 mmol of lithium hydroxide per gram.

Patent EP0662476 B1 describes the synthesis of bicyclic amidines by acid-catalysed reaction of lactones with diamines. The process is carried out in a single reaction step and is followed by purification. The patent also claims the use of these amidines as catalysts for polyurethanes. The synthesis of DBU is described in Example 6 and shows a very low product yield of 21%.

Patent publication CN1262274 A describes a method for the preparation of 1,8-diazabicyclo-[5.4.0]-undec-7-ene from ε-caprolactam and acrylonitrile, the special feature being the use of a mixture of inorganic and organic bases as catalysts in the first reaction step (KOH and DBU). The cyano-derivative obtained is subjected to purification before reduction. The second hydrogenation step is carried out in the presence of activated Ni (the catalytic form is not defined) as a catalyst but it is not mentioned whether in the presence or absence of ammonia. Dehydration is always carried out under acidic conditions, using p-toluenesulphonic acid, and in the absence of a solvent; the reaction is carried out over a fairly long period, i.e. between 35 and 40 hours, obtaining a yield for this step of 74.61%.

In all the processes described above, reference is mostly made to reductions of nitriles using Raney catalysts (Cobalt or Nickel) and in the presence of ammonia. From the literature cited, only in two documents is ammonia not used, but either a Raney catalyst, or “sponge”, is used, or numerous chemicals (including gaseous HCl) are introduced, in the latter case considerably complicating the process.

Raney catalysts are produced by treating a 50/50 Ni/Al or Co/Al alloy with an NaOH solution. In this way, most of the aluminium present is removed, giving nickel the characteristic porous “sponge” structure of Raney catalysts. Once the catalyst has been obtained, it must be stored in water or usually ethyl alcohol. In their dry activated form, Raney catalysts are pyrophoric. This makes handling the catalyst complex and introduces safety issues during the loading and unloading thereof. In addition, anhydrous ammonia is always used in reactions to reduce nitriles to amines.

The purpose of ammonia is to prevent the formation of secondary and tertiary amines when the product of interest is the primary amine. Secondary and tertiary amines originate from secondary reactions and, if not of interest for synthesis, represent for economic purposes a loss of material as well as a problem of reallocation to the market or disposal. Ammonia, in its anhydrous form, is a toxic gas and, as such, requires a great deal of care in its handling, resulting in complex plant solutions, with inevitable increases in investment and operating costs. In addition, in some countries, such as Italy, there is specific legislation regulating the use, storage and transport of toxic gases (including anhydrous ammonia); its use must therefore be authorised in accordance with this legislation, which usually takes the form of both technical and management requirements.

The patent publication CN112316949A describe the reduction of N-(2-Cyanoethyl) caprolactam by hydrogen using a catalytic system indicated like a Nickel Alloy, supported on coal, also including Cr and Fe in order to reduce the formation of primary and secondary amines. The Cr is inserted into the catalyst in Cr²⁺ form, through use of the salt of Cr(NO₃)₂ whom is extremely unstable; is not possible, indeed, to obtain it in stable form (like described by N. N Greenwood e A. Earnshaw in “Chemistry of the Elements” Vol. II page 1238 Piccin Editore 1991) due to internal redox reactions that occur during the synthesis.

For the above reasons the process like that described in the patent publication CN112316949A is not easily feasible on an industrial scale because the Cr(NO₃)₂ salt, used like precursor for obtaining the ion Cr²⁺, is so unstable that it is not commercially available.

The patent publication CN 1546492 described a method to prepare DBU (1.8-diazabicyclo(5,4,0)-7 undecene) by hydrogenation reaction in presence of a catalyst based on Al, Ni, Fe, and Cr in slurry form starting from reaction between caprolactam and acrylonitrile, with toluene like solvent, and in presence NaOH like catalyst to obtain N-(2-Cyanoethyl) caprolactam. The N-(2-Cyanoethyl) caprolactam is reduced by hydrogenation reaction to N-(3-aminopropyl) caprolactam; this last one is dehydrated to give DBU. The reactions occur by changing the type solvent between one phase and another.

The hydrogenation catalyst is obtained by lye an alloy of Ni, Al, Cr, Fe; this operation is the same method used to produce Raney or Sponge catalyst from Ni or Co alloy. For this reason the process described in the patent publication CN 1546492, leads to synthesis of a Raney or Sponge type catalyst which has the same drawbacks of the processes that use these types of catalysts.

### SUMMARY

The aim of the present disclosure is therefore the realisation of an innovative process for the synthesis of amidines which avoids the use of pyrophoric catalysts and the addition of further toxic reagents such as ammonia, while still obtaining amidine yields of industrial interest.

In particular, the present disclosure provides a process to prepare 1,8-Diazabicyclo-[5.4.0]-undec-7-ene (DBU), usable in the applications described above, from ε-Caprolactam and acrylonitrile, limiting the number of intermediate purifications and avoiding the use of anhydrous ammonia and Raney catalysts.

The Applicant therefore set out to find a process for the production of amidines from lactams and α,β unsaturated nitriles.

The Applicant has now found a method for the preparation of amidines from lactams and α,β unsaturated nitriles, successively comprising the following reaction steps: addition of the α,β unsaturated nitrile to the lactam, reduction of the cyano-derivative thus obtained in the absence of ammonia and in the absence of a Raney-type catalyst, dehydration/cyclization of the amine compound thus produced to obtain the amidine, which can finally undergo a final separation and purification step to obtain the product in a form suitable for industrial use. This method can be conducted in batch or continuous mode; continuous mode is preferred.

Surprisingly, in fact, the Applicant has found that it is possible to conduct the above reactions in series, without the use of ammonia and Raney-type catalysts, carrying out a single final purification step without the process presenting any critical issues, or requiring separation steps of the intermediates of the desired product from the other reaction products, to guarantee an acceptable final purity of the desired product and a high yield and conversion into the desired product in each of the intermediate steps. This simplifies the number of devices to be used and considerably reduces the complexity of the overall process.

Optionally, the use of intermediate purification steps can be considered if high-purity semi-finished products and/or chemical intermediates are required.

### DETAILED DESCRIPTION

These and other purposes are surprisingly achieved by the preparation process in accordance with the present disclosure.

The present disclosure provide a process for the preparation of amidines or derivatives thereof of formula (V)

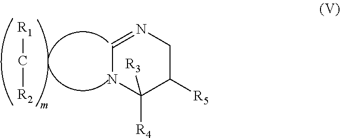

starting from a lactam having the following formula (I)

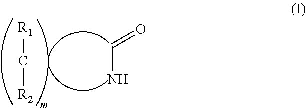

and an α,β unsaturated nitrile having the following formula (II)

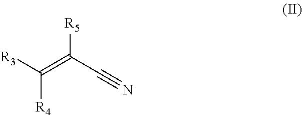

wherein:
R1 is H or an aliphatic hydrocarbon group, optionally substituted, having from 1 to 5, preferably from 1 to 2 carbon atoms, and is more preferably H;
R2 is H or an aliphatic hydrocarbon group, optionally substituted, having from 1 to 5, preferably from 1 to 2 carbon atoms, and is more preferably H;
R3 is H or an aliphatic hydrocarbon group, optionally substituted, having from 1 to 5, preferably from 1 to 2 carbon atoms, and is more preferably H;
R4 is H or an aliphatic hydrocarbon group, optionally substituted, having from 1 to 5, preferably from 1 to 2 carbon atoms, and is more preferably H;
R5 is H or an aliphatic hydrocarbon group, optionally substituted, having from 1 to 5, preferably from 1 to 2 carbon atoms, and is more preferably H;
   m is an integer from 3 to 7, more preferably from 3 to 6;
wherein, even more preferably, (I) is ε-Caprolactam, (II) is acrylonitrile, and (V) is 1,8-Diazabicyclo [5.4.0] undec-7-ene (DBU),
said process comprising the following steps in sequence:
(A) reacting under addition conditions, with one of the methods known to those skilled in the art, said compound of formula (I) with said compound of formula (II) in the presence of a suitable basic catalyst, preferably KOH, NaOH, LiOH and tetrabutylammonium hydroxide, obtaining a compound of formula (III):

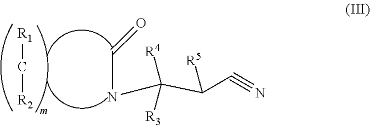

NaOH, LiOH and tetrabutylammonium hydroxide are preferred. In another way, said basic catalyst is preferably DBU, primary amines, secondary amines, tertiary amines or other organic hydroxides;
(B) subjecting said compound of formula (III) as obtained in step (A), preferably in the absence of intermediate steps of purification of the compound from the other reaction products, to reduction by reaction with hydrogen in the presence of a catalyst based on metals of groups 8, 9 and 10 of the periodic table, such as, for example, Iron, Cobalt, Nickel, or noble metals such as Ruthenium, Rhodium, Palladium, Osmium, Iridium or Platinum, wherein said catalyst is not a Raney or sponge type catalyst, to obtain the corresponding amine of formula (IV) (primary amine) and optionally separate it from the reaction solvent;

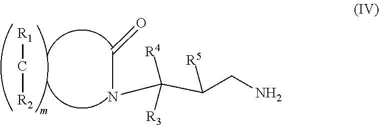

(C) subjecting said amine to dehydration, by one of the methods known to those skilled in the art, obtaining the corresponding amidine of formula (V). The amidine of formula (V), synthesised as described above in accordance with the present disclosure, may be subjected to solvent recovery and subsequent purification.

In accordance with the present disclosure, the term amidine means a compound derivable from an amide by substitution of the oxygen of the carbonyl group ═CO by an imide group ═N—. Preferably, the present disclosure also considers cyclic amidines as defined in formula (V).

In accordance with the present disclosure, the term “amidine derivative” means any compound obtainable from an amidine by reaction with a carboxylic acid, an epoxyketone, chloroformates or carbonic acid diesters.

In accordance with the present disclosure, the singular indefinite article, one, is understood to include also the meaning of at least one, unless otherwise specified.

The present disclosure provides a process to synthesize an intermediate of formula (IV) starting from a compound of formula (III) as defined above, for the preparation of intermediates usable for the synthesis of amine derivatives comprising an N-alkyl-lactam chain.

In accordance with step (A) of the process according to the present disclosure, a controlled catalytic addition reaction is conducted to obtain the compound of formula (III) with high yields starting from a lactam of formula (I), preferably ε-caprolactam, and an α,β unsaturated nitrile of formula (II), preferably acrylonitrile, in the presence of a suitable basic catalyst.

The molar ratio (II)/(I) is chosen by the person skilled in the art according to what is known in organic chemistry for step A reactions, preferably between 1.4 and 0.7, more preferably between 0.8 and 1.3, for example about 1.1.

In this document, unless otherwise indicated, percentages are to be understood as percentages by mass.

In this document, unless otherwise indicated, pressures are to be considered absolute.

Typically, the reaction is carried out at temperatures of 20 to 140° C. and pressures of 0.1 to 6 barA, for times that can range, depending on the type of reagents (I) and (II), temperature and pressure, from 0.5 to 10 hours, preferably 0.8 to 4 hours. According to a preferred mode, the pressure is atmospheric. In another preferred mode, the pressure is above atmospheric, preferably 1.1 to 6 barA.

The reaction may be carried out on compounds of formula (I) and (II) in the absence of solvent or in the presence of a suitable quantity of organic solvent, preferably between 5 and 70% by weight of the total of said reaction mixture.

Said solvent may be, for example, a polar solvent such as a linear, branched or cyclic ether, for example, methyl tert-butyl ether (MTBE), tetrahydrofuran (THF), or an alcohol having 1 to 6 carbon atoms such as methanol or ethanol, isopropyl or tert-butyl alcohol, or an aromatic solvent such as benzene, toluene, xylenes, ethylbenzene, or an aliphatic hydrocarbon such as heptane or cyclohexane.

Preferably the solvent is chosen from among the above classes of compounds in such a way that it is capable of solubilising the compound (III) in the reaction environment. In addition, the solvent preferably has a lower boiling point than the compounds of formula (I) and (III) so that it can be separated from these, at least in part, by evaporation.

Preferred solvents are isopropyl alcohol, tert-butyl alcohol, MTBE, THF, toluene, ethylbenzene, xylenes.

In accordance with this process, all bases known in the literature and suitable for the purpose can be used as catalysts.

According to one mode, said catalyst may be an inorganic base, preferably KOH, NaOH and LiOH, or an organic hydroxide, preferably tetrabutylammonium hydroxide. In another way, said catalyst may be an organic base, preferably DBU, primary, secondary, tertiary amines or other organic hydroxides.

Compared to the prior art, step (A) is distinguished by favourable operating conditions in terms of reaction mixture composition and reaction time, which are elementary operations necessary to obtain good yields of the product of interest when coupled with the innovative step (B).

The compound of formula (III) obtained in step (A) can be separated and purified from the reaction mixture containing it, which comprises the by-products, the catalyst and/or its residues and the possible solvent.

The Applicant has, however, surprisingly found that such separation and purification step of the intermediate compound of formula (III) from the other reaction by-products may not be carried out if the next step is a reduction conducted as in step (B). However, a partial evaporation of the solvent can optionally be carried out to avoid excessive dilution. This avoids costly separation and purification of the by-products.

In the subsequent step (B) of the process according to the present disclosure, the intermediate of formula (III) coming from step (A), preferably without being separated from the reaction mixture, except possibly by partial evaporation of the solvent, is subjected to reduction to convert it into the corresponding amino derivative of formula (IV).

The reduction of nitriles is a reaction already known and reported in literature and widely used in organic synthesis (see, for example, Peter Vollhardt, Organic Chemistry p. 825-826 1st Edition). In the patents listed above, it is conducted on a compound of formula (III) using Raney catalysts (Ni or Co) or otherwise in sponge form, and in the presence of anhydrous ammonia.

In some patent applications of the known art such reduction is carried out on catalyst indicated like “alloy”, for example “nickel alloy catalyst”, where the “alloy” is a compound in which two or more metals have been dissolved in each other, in the molten state, so as to create an intimate combination between metals: the catalyst according to the present disclosure also exclude this type of “alloy” catalysts and are not identifiable with them.

For the “alloy” definition it's possible to refer to as is defined in known scientific books and/or manuals like, for example, the book of Alan Cottrel “An Introduction to Metallurgy” (chapter 14, pag. 189), second edition 1995—The Institute of Materials London.

Suitable reduction catalysts for the purposes of the present disclosure are commercial or synthetic hydrogenation systems based on one or more metals of groups 8, 9 and 10 of the periodic table, such as, for example, Iron, Cobalt, Nickel, or noble metals such as Ruthenium, Rhodium, Palladium, Osmium, Iridium or Platinum. Cobalt, Nickel, Palladium and Platinum are preferred. Cobalt and Nickel are particularly preferred. Such catalysts can be used in the dispersed, colloidal or in solid phase supported/bonded form, preferably in solid phase supported/bonded form on the inorganic phase with a high surface area, even more preferably in the supported/bonded phase on silica, alumina or silica-alumina, with the exclusion of pyrophoric catalysts in metallic sponge form, such as Nickel Raney or Cobalt Raney and metal catalysts defined as “alloys” which are not part of the present disclosure. Cobalt on an alumina support is particularly preferred.

Generally, these types of catalysts according to the present disclosure are obtained by means of various techniques, starting from aqueous solutions of precursor salts; therefore they are not metals alloys.

In a preferred embodiment, reduction catalysts are Co- and Ni-based catalysts, preferably supported/bonded on a Lewis acid or a Lewis acid having Bronsted acid components, more preferably Al₂O₃ or SiO₂, wherein said catalyst is not a Raney or sponge-type catalyst.

In accordance with step (B) of the present process, therefore, the reduction of the compound of formula (III) is conducted using said reduction catalyst, with H₂ and in the absence of ammonia, in the presence of water in a molar ratio of H₂O/(III) comprised between 0.01 and 1 or in a weight ratio of between 0.1 and 11% with respect to the reagent mixture.

The reaction temperature of step (B) is comprised between 30 and 250° C., preferably between 50 and 200° C., and the pressure is comprised between 4 and 150 barA, preferably between 11 and 100 barA, even more preferably between 20 barA and 60 barA.

The reduction reaction may be conducted in batch (in a reactor equipped with a stirrer, heating jacket and inlets for gases and liquid streams) for a reaction time of 0.1 to 12.0 h, preferably comprised between 0.8 and 7.0 h, more preferably from 1.5 to 5 h; or, it may be conducted continuously, e.g. in a single- or multi-stage tubular reactor or in a stirred reactor such as a CSTR. Continuous mode is preferred for productivity issues, particularly on an industrial scale.

The reduction reaction can be carried out in the presence of an organic solvent. In one embodiment, said organic solvent is preferably chosen from methanol or ethanol, isopropyl or tert-butyl alcohol, MTBE, THF, more preferably THF. In another embodiment, the organic solvent is an aromatic solvent such as benzene, toluene, xylenes, ethylbenzene, most preferably xylene (o, m, p or mixture of isomers) and ethylbenzene.

In the preferred case where the compound of formula (III) is a cyano-derivative of ε-caprolactam, the main product of the reduction is the corresponding amino-derivative (IV), predominantly obtained together with a quantity of the cyclized product (V) (DBU).

There may also be the formation of the corresponding secondary/tertiary amines. However, these compounds are obtained in quantities in line with those found in literature when ammonia is used, and in any case in negligible quantities compared to the desired primary amine.

The amino-derivative of the lactam of formula (IV), obtained in step (B) of the present process, is subjected to dehydration through step (C) for the synthesis of the corresponding amidine, in the preferred case DBU (1,8-Diazabicyclo [5.4.0] undec-7-ene).

For step (C) the Applicant used what is already described in the prior art, in particular what is described in German patent DE1545855.

The dehydration is carried out hot, preferably between 90 and 270° C., more preferably between 130 and 230° C., even more preferably between 150 and 200° C., continuously removing the water produced during the dehydration which operates the cyclization; it is possible to operate in boiling and partial condensation of the vapours in a reflux mode, collecting the condensates in a phase separator where the water is separated and the solvent refluxed within the reaction system.

The acid catalyst is always necessary and can be chosen by a person skilled in the art from those known in literature, in the case of the present disclosure p-toluenesulphonic acid. At the end of the reaction of step (C), the mixture must be neutralised with the appropriate amount of a concentrated aqueous NaOH solution, and the solvent is finally recovered by evaporation. The main dehydration product is the amidine of interest.

If required by end-users, the amidine can be purified by one of the methods already known in the state of the art, e.g. by distillation to a purity which varies from 95 to 98% by weight.

The process according to the present disclosure is therefore advantageous because it eliminates the problems related to the pyrophoricity of Raney catalysts and the toxicity of ammonia, without penalizing the formation of the primary amine; moreover, the Applicant has surprisingly noted the formation of amidine already in step (B).

None of the methods described above in the prior art mentions the possibility of carrying out a reduction of nitriles in the absence of ammonia and Raney catalyst.

Preferably, in the process of the present disclosure, no intermediate purification of the reaction mixtures obtained in steps A) or B) is carried out, but only the evaporation of any solvent for the recovery and use thereof.

The Applicant also surprisingly identified the possibility of using a single solvent for all the reaction steps, further simplifying the process.

This solvent can be chosen from the class of aprotic solvents; in particular, the use of xylene (pure isomers or mixtures) has proved particularly suitable for this purpose.

In this process, all the reaction steps and the final purification step can be conducted continuously.

In particular, the use of a single solvent for all the reactions further simplifies the process, making it even more efficient in terms of productivity and operating costs in the continuous configuration.

In a particularly preferred embodiment of the present disclosure, the Applicant has found a novel and original process for producing amidines from lactams.

The process in accordance with the present disclosure is therefore described below in greater detail with reference to the production of 1,8-Diazabicyclo [5.4.0] undec-7-ene (DBU), starting from ε-caprolactam and acrylonitrile without, however, this being in any way understood as limiting the application of the same inventive process to compounds having a different structure and different number of carbon atoms, within the limits of formulae (I) and (II) above.

The mixture of compound (I) (e.g. ε-Caprolactam) in a solvent (e.g. xylene) is continuously fed, after addition of the basic catalyst (e.g. NaOH, LiOH or tetrabutylammonium hydroxide), to a CSTR or tubular recirculating reactor into which compound (II) (e.g. acrylonitrile) is also continuously fed. A further preferred solution is to have two reactors with these characteristics placed in series. Alternatively, it is possible to use the reactor in batch mode, sending the reaction product into a tank from which it is continuously sent to step (B). The addition reaction takes place at a temperature between 20 and 140° C., preferably between 40 and 110° C., even more preferably between 60 and 80° C., with a residence time of 0.5 to 10 h, preferably between 0.8 and 4 h.

Compound (I) (such as ε-caprolactam, for example) can also be fed molten in the absence of solvent, although it is the preferred solvent mixture. In the latter case, said solvent may be present up to 70% by weight of the whole solution, preferably 5 to 50%, more preferably 15 to 40% by weight of the whole solution.

The pressure at which the reaction is conducted is comprised between 0.1 and 6 barA, preferably between 0.1 and 4 barA.

Since the reaction is exothermic, the reaction temperature can also be controlled by partial evaporation of the reaction mixture with reflux condensation in the reactor; alternatively, the reaction mixture can be recycled through an external heat exchanger on the reactor itself.

The output and conversion of step (A) are typically high. For example, in the case where compound (I) is ε-caprolactam and compound (II) is acrylonitrile, the main addition product, N-(2-Cyanoethyl)-ε-caprolactam, is obtained in yields typically up to 90-95%, while the conversion of ε-caprolactam is typically between 85 and 99.9%.

All conversion, selectivity and yield values mentioned refer to those determined by ¹H and ¹³C NMR and GC-MS analysis of the reaction mixtures as described in the examples.

The stream leaving the reactor is optionally cooled (with the possibility of partial heat recovery) or sent directly to the second step (B) of the reduction reaction.

Alternatively, the liquid stream may possibly be fed to an evaporator to recover the solvent and any reagents, i.e. unreacted compounds (I) and (II). Any type of evaporator known in the prior art may be advantageously used for the purpose of the present disclosure. Preferably, a kettle-type evaporator is used. More details on the types of evaporators that can be used for this purpose can be found, for example, in Perry's Chemical Engineers' Handbook, McGraw-Hill (7th Ed.-1997), Section 11, pages 108-118. An alternative set-up is based on the use of a flat distillation column or with fillers. The distillation column makes it possible to recycle any unreacted compound (I) and compound (II) or solvent, with a lower content of reaction products than when using an evaporator.

The liquid stream leaving the evaporator, or the bottom of the distillation column, containing the addition products and the solubilised catalyst, is then sent to an exchanger and heated to a temperature of between 30° C. and 250° C., preferably between 50° C. and 200° C., more preferably between 100° C. and 160° C.; said stream coming from said exchanger is sent to a reactor for the reduction reaction; said reactor is preferably a fixed bed reactor, or trickle bed reactor, operating at a WHSV (Weight Hourly Space Velocity, related to the sum of all the incoming streams) comprised between 1 and 50 h⁻¹, preferably between 3 and 10 h⁻¹. This reactor is equipped with a thermostat system and contains a hydrogenation catalyst as described above.

The reduction reaction can be carried out in the presence of an organic solvent, preferably chosen from MTBE, THF, methanol, ethanol, isopropanol, tert-butyl alcohol, or toluene, xylene (pure or mixed isomers), ethylbenzene. THF, xylene and ethylbenzene are preferred and the solvent is preferably the same as that used in step (A). Said solvent may be present from 3% to 70% by weight of the reaction mixture, preferably from 5 to 50% by weight, more preferably from 15 to 40% by weight of the reaction mixture.

The reduction reaction is preferably carried out in the presence of water, in an amount comprised between 0.1 and 11% by weight of the reagent mixture; said reactor is fed with H₂ to a pressure comprised between 4 and 150 barA, preferably between 11 and 100 barA, more preferably between 20 and 60 barA. The reactor is continuously flushed with gas by recycling the outgoing gas from the reactor head to the bottom of the reactor via a compressor/fan. A portion of reintegrated H₂ is supplied to maintain the pressure values indicated above. A stream comprising the mixture of reaction products and, optionally, the solvent flows from the bottom of the reactor. A preferred set-up of this reactor envisages recycling excess gas by means a liquid jet ejector which is installed on top of a trickle bed reactor. The motor fluid is the same reaction mixture that is recirculated through a pump.

If compound (I) is ε-caprolactam and compound (II) is acrylonitrile, the main reduction product is N-(3-Aminopropyl)-ε-caprolactam and possibly also 1,8-Diazabicyclo-[5.4.0]-undec-7-ene (DBU); the main by-products are the secondary and tertiary amines of 3-aminopropyl-ε-caprolactam. These by-products do not exceed 7% by weight. The conversion of N-(2-cyanoethyl)-ε-caprolactam is between 90 and 99%, the overall yield of N-(3-aminopropyl)-ε-caprolactam and DBU is greater than 92%. All conversion, selectivity and yield values mentioned refer to those determined by ¹H and ¹³C NMR and GC-MS analysis of the reaction mixtures.

This current can be sent to a solvent recovery system. The preferred set-up is one based on an evaporator for water and solvent recovery. The reaction mixture with traces of solvent comes out of the bottom of the evaporator. The steam from the evaporator is fed to the degasser, which contains perforated plates to facilitate both the separation and contact of the two phases, liquid and steam. The vapour phase leaving the degasser is partially condensed in a reflux condenser, which operates at a temperature of 20-250° C., preferably 40-150° C., even more preferably at 60-130° C.; optionally, further condensation can be carried out to recover any by-products formed during the reactions of step (A) and (B).

The vapours leaving the reflux condenser are condensed in another condenser at a temperature comprised between 2-50° C. preferably 10-30° C., more preferably 20° C.

The liquid that collects at the outlet of the condenser is solvent plus water; after separation of the water, the solvent is recycled, while the mixture coming out of the bottom of the evaporator can be sent to the dehydration step (C).

However, in a preferred embodiment, this stream leaving the hydrogenation reactor is sent directly to the dehydration step.

The dehydration/cyclization of N-amino lactams is a reaction known from literature that can be carried out in a variety of ways by a person skilled in the art. The following methods refer to the conditions employed by the Applicant and are in no way to be considered as limiting the process of the present disclosure.

Dehydration takes place continuously in a reactor, called a dehydrator, preferably of the CSTR type, equipped with a heating system and a condensing system comprising a partial reflux condenser and a post-condenser which condenses most of the water produced and sends the condensates to a phase separator where the solvent is reintroduced. In the phase separator, any traces of organic matter are separated and reintroduced into the dehydrator, while the water can be partly recycled to the hydrogenation section and the excess sent for treatment. The reaction is conducted in the presence of a solubilised acid catalyst in the reaction environment, preferably p-toluenesulphonic acid, for a residence time of between 0.5 and 12 h, preferably between 2 and 8 h. Optionally, the reaction can also be carried out in the absence of a solvent. The dehydration is carried out hot, preferably between 90 and 270° C., more preferably between 130 and 230° C., even more preferably at the boiling point of the mixture. The pressure at which the reaction is conducted is between 0.08 and 5 BarA, preferably between 0.5 and 3 BarA, more preferably between 1 and 2 BarA. At the end of the reaction, the mixture must be neutralised with the appropriate amount of concentrated aqueous solution of a strong base such as NaOH, and the salt formed must be removed from the mixture. A stream of dehydration products, solvent, any unreacted amine and by-products from the previous steps exits from the bottom of the reactor; if compound (I) is ε-caprolactam and compound (II) is acrylonitrile, the main product is DBU (1,8-diazabicyclo [5.4.0] undec-7-ene).

Said stream is then sent to a distillation section for solvent recovery and purification of the compound (V), such as DBU. After distillation, the purity of this compound is typically comprised between 95 and 98%.

The purity of said compound is determined by gas chromatography analysis (GC-MS).

Optionally, said compound after distillation can be subjected to further purification such as liquid-liquid extractions. Such operations can be carried out using techniques known to a person skilled in the art.

According to a different embodiment of the disclosure, the dehydration/cyclization reaction of the amine of formula (IV) can advantageously be conducted with an alumina, silica alumina or zeolite catalyst obtaining the corresponding amidine of formula (V).

The amidine of formula (V), synthesised as described above in accordance with the present disclosure, may be subjected to subsequent purification by methods known to a person skilled in the art. In this embodiment, the reaction mixture from the hydrogenation step B) is preferably subjected to solvent recovery by evaporation and then to dehydration. Alternatively, although in a less preferable embodiment, the amino derivative of the lactam of formula (IV) can be reacted in purified form. In yet another embodiment, dehydration can be conducted in the same solvent as in the previous steps, e.g. xylenes.

The dehydration is carried out hot, preferably between 90 and 270° C., more preferably between 130 and 230° C., even more preferably between 150 and 200° C., continuously removing the water produced during the dehydration process which operates the cyclization.

The catalyst is always required and is chosen from heterogeneous acid catalysts chosen from Lewis acids or Lewis acids with Bronsted acid components, such as aluminium oxide (γ-Al₂O₃), alumina silica (SiO₂—Al₂O₃), acid earths such as lanthanum oxide and zirconium oxide, or heterogeneous catalysts based on resins, such as sulphonated resins or ion exchange resins. Said catalysts may be supported on inert carriers such as pumice, graphite or silica. Aluminium oxide (γ-Al₂O₃) is preferred. At the end of the reaction the main dehydration product is the amidine of interest of formula (V). If required by end-users, the amidine can be purified by one of the methods already known in the state of the art, e.g. by distillation to a purity which varies from 95 to 98% by weight.

The Applicant has therefore surprisingly identified the possibility of operating solvent-free dehydration on solid acid catalyst without refluxing the solvent, in order to facilitate water removal, with further simplification of the process and reduction of costs.

In the present process, the reaction step (C) and the final purification step can be conducted continuously.

Dehydration takes place continuously in a reactor, called a dehydrator, preferably of the tubular type, equipped with a heating system and a condensing system comprising a post-condenser which condenses most of the water produced and sends the condensates to a phase separator. In the phase separator, any traces of organic matter are separated and reintroduced into the dehydrator, while the water can be partly recycled to the hydrogenation section and the excess sent for treatment. In a preferred form, the mixture is continuously fed laterally into the reactor, while steam comes out of the reactor head and the reaction product from the bottom. This reactor may optionally contain fillings in the upper part, such as rings, plates, septa, so that only water vapour can escape. In another embodiment, the reaction mixture can also be continuously fed from the bottom and the reaction product taken from the side of the reactor while water vapour escapes from the reactor head. The reaction is conducted in the presence of a heterogeneous acidic catalyst, preferably γ-alumina, with a WHSV (Weight Hourly Space Velocity, relative to the entire reagent mixture) comprised between 1 and 50 h⁻¹, preferably between 3 and 10 h⁻¹. The dehydration is conducted hot, preferably between 90 and 270° C., more preferably between 130 and 230° C., even more preferably between 150 and 200° C. The pressure at which the reaction is conducted is comprised between 0.08 and 5 BarA, preferably between 0.5 and 3 BarA, more preferably between 1 and 2 BarA. A stream comprising the dehydration products, any unreacted amine and eventually solvent, and the by-products of the previous steps exits from the bottom of the reactor; where the compound of formula (IV) is N-(3-aminopropyl)-ε-caprolactam, the main product is typically DBU (1,8-Diazabicyclo [5.4.0] undec-7-ene).

Said product stream is then sent to a distillation section for solvent recovery and purification of the compound (V), such as DBU. After distillation, the purity of said compound is typically comprised between 95 and 98%.

The purity of said compound is determined by gas chromatography analysis (GC-MS).

### EXAMPLES

In the following examples, the following abbreviations and materials are used, unless otherwise indicated:
AN: acrylonitrile (CAS 107-13-1, purity≥99%, Sigma-Aldrich)
CPLT: ε-caprolactam (CAS 105-60-2, purity 99%, Sigma-Aldrich)
NaOH: sodium hydroxide (CAS 1310-73-2, purity≥98%, Sigma-Aldrich)
Aqueous solution of NaOH min. 45% (CAS 1310-73-2, titre 45-50%, Sigma-Aldrich)
Xylene: mixture of xylene isomers (CAS 1330-20-7, purity≥98.5%, Sigma-Aldrich)
CTZ1: commercial catalyst HTC CO 2000 RP 1.2 mm (Co≈15% supported on alumina) Johnson-Matthey (data from U.S. Pat. No. 8,293,676 B2 Table 3 columns 21-22 Example J)
CTZ2: commercial catalyst HTC Ni 500 Johnson-Matthey (in the form of a 1.2 mm trilobite extruded material containing 21% nickel as nickel oxide, on a porous transition alumina support, data from international patent application (PCT) WO 2010/018405 example 1 on page 6)
H₂: hydrogen (Sapio Titre 5.5)
H₂O: ultra-pure water (MilliQ millipore system)
p-TSA: p-toluenesulphonic acid monohydrate: (CAS 6192-52-5, purity 99%, Sigma-Aldrich)

### Gas-Mass Analysis

The gas-mass analysis for the determination of reagents and reaction products of the three reaction steps is carried out with a GC HP6890 chromatograph, equipped with a split/splitless injector and interfaced to an MS HP 5973 mass spectrometer acting as a detector. The chromatograph features an HP-1MS UI capillary column (100% polydimethylsiloxane, Agilent J&W), fused silica WCOT, 30 m length, 0.25 mm ID, film thickness 0.25 μm. The instrumental parameters are as follows:
injection volume 20 μl
Helium carrier gas at 0.8 ml/min (constant flow mode)
splitting ratio 250:1
Injector temperature 300° C.
programmed oven temperature from 40° C. to 320° C. at 10° C./min (28 min) plus hold time of 10 min at 320° C. (total run time=38 min).

In the absence of the market availability of certain pure products (such as the cyano derivative of ε caprolactam and the corresponding amine), quantification was performed by the method of comparing the relative areas of the various chromatography peaks (thus accepting the approximation that they all have the same chromatography response).

However, a quantitative ¹H e ¹³C NMR analysis was also carried out on the same samples, and the results obtained were superimposable on those shown with the gaschromatographic technique.

### NMR Analysis

Analyses on the samples provided were carried out using a Bruker Avance 400 MHz spectrometer at a temperature of 300 K by dissolving approximately 50-70 mg of sample in deuterated chloroform. The spectra were recorded with the following instrumental parameters:

| | PROBE: 5 mm PABBO BB/19F-1H/D Z-GRAD | | | |
|---|---|---|---|---|
| | ¹H - 400 MHz | | ¹³C - 100 MHz | |
| Method | zg30 | | zgpg30 | |
| No. of scans | 64 | | 512 | |
| No. of data points | 32k | | 32k | |
| p1 (μs) | 26.00 | | 17.00 | |
| d1 (s) | 7.00 | s | 8.00 | s |
| Spectral window | 15 | ppm | 240 | ppm |
| O1P | 4.0 | ppm | 100.0 | ppm |
| PL1 | −0.85 | dB | −0.85 | dB |

| | |
|---|---|
| Solvent | CDCl₃ 99.9 atom % D + ~0.1% TMS |

### Example 1: Reaction Between ε-Caprolactam and Acrylonitrile in Xylene

123.3 g of ε-caprolactam and 62.5 g of xylene were placed in a 1 1 flask equipped with nitrogen inlet, stirrer, reflux condenser, thermocouple and dropping funnel. Under a light flow of nitrogen, the suspension was heated while stirring at 45-50° C. using an oil bath; once completely solubilised, 0.1841 g of NaOH was added and the temperature was brought to 70° C. Once the sodium hydroxide was solubilised, acrylonitrile (67.4 g) was dripped, taking care to maintain the temperature between 70 and 80° C.; the reaction was exothermic (estimated addition time about 1 h). At the end of the addition of the acrylonitrile, the temperature was kept at 70° C. and the reaction was allowed to continue for 2.25 h. A progressive darkening of the solution was observed as the addition reaction progressed.

GC-MS analysis showed a caprolactam conversion of 98.6%, a selectivity of 98.3% and thus a product yield of 96.9%. The basic raw solution was subjected to hydrogenation as described below in example 2.

### Example 2: Hydrogenation of Crude Nitrile Solution in Xylene (Cat. Co)

In a 250 ml autoclave equipped with a mechanical turbine stirrer, heating mantle, catalyst basket, inlet for gases and liquid streams, 30 g of CTZ1 catalyst was introduced at room temperature into the dedicated catalyst basket and activated in a hydrogen atmosphere.

Activation of the catalyst was carried out by first flushing it with nitrogen at atmospheric pressure, after which the reactor was heated to 150° C. with a temperature ramp of 25-50° C./h, and once this temperature had been reached, hydrogen was supplied at a flow rate of 30 ml/min, thus raising the temperature to 180° C.

At this point, the hydrogen flow rate was increased by progressively reducing the nitrogen flow rate until the gas flushing was completely hydrogen-based (flow rate 200 ml/min). Under these temperature and flow rate conditions, activation continued for 18 hours, after which the nitrogen current was restored (and at the same time the hydrogen current was reduced) in order to maintain the catalyst in an inert atmosphere, gradually cooling the system to room temperature.

To 143.9 g of the solution obtained from example 1, 4.5 g of H₂O was added (approx. 3% of the total) and then loaded into the reactor; the lines were then washed by introducing a further 20.1 g of xylene. The reactor pressure was raised to 21 barA by driving the stirrer motor (750 rpm) and turning on the heater, setting an internal temperature of 130° C. In the meantime, the reactor was pressurised with hydrogen to a pressure of 41 barA. It was hydrogenated at this pressure as long as there was a hydrogen flow of about 0.2-0.3 L/h in line towards the reactor. The volume of hydrogen introduced into the reactor was also indicated by means of the counter and compared with the stoichiometric quantity calculated based on the amount of nitrile introduced. Finally, the product was cooled and discharged.

GC-MS analysis showed a conversion of the nitrile product of 96.1%, a selectivity of 99.3% and thus a yield in the products N-(3-aminopropyl)-ε-caprolactam and DBU (1,8-Diazabicyclo [5.4.0] undec-7-ene) of 95.4%. The synthesis was reproduced under the same conditions 2 more times in order to obtain a sufficient quantity of product to be dehydrated (the results obtained are superimposable with those presented in this example).

### Example 3: Dehydration of Crude Amine Solution in Xylene

To 159.4 g of solution obtained as described in example 2, 1.3 g of p-toluenesulphonic acid monohydrate was added. This mixture was heated to 150-160° C. by reflux through the use of a dry-half spherical heater connected to a temperature controller; the reaction flask was connected to a Dean-Stark trap and a condenser to remove the water produced from the reaction environment: the vapours produced condensed on top of the trap and the water thus formed was removed by gravity into the trap (the solvent fell back into the flask maintaining an almost constant volume). After 4 h from the start of reflux, no further accumulation of water was observed in the trap and the reaction was considered to be complete, then allowed to cool to room temperature. The resulting solution was neutralised with 800 mg of aqueous NaOH solution (min. concentration 45%).

GC-MS analysis calculated a conversion of N-(3-aminopropyl)-ε-caprolactam of 92.3%, a selectivity of 99.4% and thus a DBU yield of 91.7%.

### Example 4: Hydrogenation of Crude Nitrile Solution in Xylene (Cat. Ni)

The same reaction as described above in example 2 was carried out substituting the catalyst CTZ1 with the catalyst CTZ2 (the activation mode is the same to that already described above).

GC-MS analysis showed a conversion of the nitrile product of 96.9%, a selectivity of 78.5% and thus a yield in the products N-(3-aminopropyl)-ε-caprolactam and DBU (1,8-Diazabicyclo [5.4.0] undec-7-ene) of 76.1%.

### Example 5: Reaction Between Caprolactam and Acrylonitrile in the Absence of a Solvent

The same reaction as described in example 1 was also carried out in the absence of a solvent.

123.4 g of ε-caprolactam were placed in a 500 ml flask equipped with nitrogen inlet, stirrer, reflux condenser, thermocouple and dropping funnel. Under a light flow of nitrogen, the solid was heated to 70-75° C. using an oil bath (external temperature control); when fully melted, 0.1230 g of NaOH were added and the temperature was raised to 70° C. (internal temperature control). Once the sodium hydroxide was solubilised, acrylonitrile (67.4 g) was dripped, taking care to keep the temperature between 70 and 80° C. The reaction was exothermic. At the end of the addition of the acrylonitrile, the temperature was kept at 70° C. and the reaction was allowed to continue for 2 h. A progressive darkening of the solution was observed as the addition reaction progressed.

GC-MS analysis showed a caprolactam conversion of 95.4%, a selectivity of 98.9% and thus a product yield of 94.4%.

### Example 6: Dehydration of Crude Amine Solution in Xylene with Heterogeneous Acid Catalyst

The solution from the example 2 (138.3 g) was introduced into a flask (containing a few glass balls), which was connected to a Dean-Stark apparatus equipped with a bubble cooler. One gram of SASOL SPHERES 1.0/160 alumina, previously activated in an oven at 150° C. for 8 hours, was then added. The flask was heated to 170° C.; the water formed by the reaction was separated while the solvent was recovered. After approximately 4 h, no more water formation was observed; the flask was then cooled and the contents subjected to GC-MS analysis. The analysis resulted in a conversion of N-(3-aminopropyl)-ε-caprolactam of 94.7%, a selectivity of 99.5% and thus a DBU yield of 94.2%.

### Example 7: Amine Dehydration with Solvent-Free Heterogeneous Acid Catalyst

The solvent of the mixture obtained from the example 2 was removed with a rotavapor (T=60° C.; P=30 mbar), resulting in 113.9 g of a mixture of N-(3-aminopropyl)-ε-caprolactam and DBU; this solution was introduced into a flask (containing a few glass beads), connected to a Liebig condenser for removal of the reaction water. One gram of SASOL SPHERES 1.0/160 alumina, previously activated in an oven at 150° C. for 8 hours, was then added. Dehydration was conducted in a gentle flow of nitrogen to facilitate water removal. The flask was then heated to 170-180° C. for approximately 5 h (at which time no condensate formation was observed); the flask was then cooled and the contents were subjected to GC-MS analysis. The analysis resulted in a conversion of N-(3-aminopropyl)-ε-caprolactam of 93.6%, a selectivity of 83.1% and thus a DBU yield of 77.8%.

Tables 1, 2 and 3 show the summary data of the previous examples.

Finally, it is understood that further modifications and variations to the process as described and illustrated herein may be made which are not specifically mentioned in the text, but which are to be considered as obvious variations of the present disclosure within the scope of the appended claims.

### Calculation of Conversions, Selectivity and Yields (GC-MS)

$$conv \% = \frac{mol initial reagent - mol residual reagent}{mol initial reagent} * 100$$

$$sel \% = \frac{mol obtained product}{mol reacted reagent} * 100 yield = \frac{conv \% * sel \%}{100}$$

### Where:

mol—number of moles

TABLE 1
Addition reactions

| Example | 1 | 5 |
|---|---|---|
| CPLT (g) | 123.3 | 123.4 |
| AN (g) | 67.4 | 67.4 |
| NaOH (g) | 0.1841 | 0.1230 |
| Solvent (≈25% w/w) | Xylene | — |
| Temperature (° C.) | 70 | 70 |
| Time (1) (h) | 2.25 | 2 |
| Pressure (barg) | 0 | 0 |
| CPLT conversion (%) | 98.6 | 95.4 |
| Nitrile selectivity (%) | 98.3 | 98.9 |
| Nitrile yield (%) | 96.9 | 94.4 |

(1) Reaction time at the end of the AN dosing (dosing time in all examples is 1 h)

TABLE 2
Reduction reactions

| Example | 2 | 4 |
|---|---|---|
| Nitrile compound in | From example 1 | From example 1 |
| solution (ref.) | | |
| Nitrile compound mass(g) | 143.9 | 143.0 |
| Water (% w/w of total) | 2.67 | 2.84 |
| Solvent (≈25% w/w) | Xylene | Xylene |
| CTZ (metal supported on | 30 g CTZ1 (Co) | 30 g CTZ2 (Ni) |
| Al₂O₃, g) | | |
| Stirring speed (rpm) | 750 | 750 |
| T (° C.) | 130 | 160 |
| P H₂ (Barg) | 40 | 40 |
| Nitrile Conv. (%) | 96.1 | 96.9 |
| Select. (2) (%) | 99.3 | 78.5 |
| Yield (2) (%) | 95.4 | 76.1 |

(2) Yield and selectivity towards all desired products (primary amine plus DBU)

TABLE 3
Dehydration reactions

| Example | 3 | 6 | 7 |
|---|---|---|---|
| Amine compound in | From | From | From |
| solution (ref.) | example 2 | example 2 | example 2 |
| Primary amine/DBU | 159.4 | 138.3 | 113.9 (no |
| solution mass(g) | | | solvent) |
| Solvent | Xylene | Xylene | — |
| p-TSA mass (g) | 1.3 | — | — |
| CTZ mass (Al₂O₃, | — | 1 | 1 |
| SASOL Spheres 1.0/ | | | |
| 160, g) | | | |
| T (° C.) | 150-160 | 170 | 170-180 |
| P (Barg) | 0 | 0 | 0 |
| Primary amine conv. (%) | 92.3 | 94.7 | 93.6 |
| DBU Select. (%) | 99.4 | 99.5 | 83.1 |
| DBU yield (%) | 91.7 | 94.2 | 77.8 |

Considering the results of examples 1, 2 and 3 in succession, it was possible to calculate the overall yield of the synthesis (with p-TSA acid):

| Yield | Yield | Yield | Total |
|---|---|---|---|
| example 1 | example 2 | example 3 | yield |
| (%) | (%) | (%) | (%) |
| 96.9 | 95.4 | 91.7 | 84.8 |

1. A process for the preparation of amidines or derivatives thereof of formula (V) 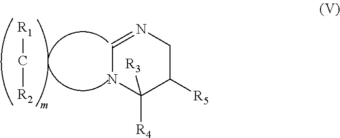
starting from a lactam having the following formula (I): 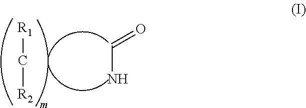
and an α,β unsaturated nitrile having the following formula (II) 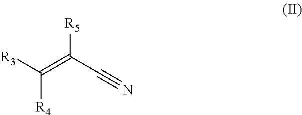
wherein:
R1 is H or an aliphatic hydrocarbon group, optionally substituted, having from 1 to 5, preferably from 1 to 2 carbon atoms, and is more preferably H;
R2 is H or an aliphatic hydrocarbon group, optionally substituted, having from 1 to 5, preferably from 1 to 2 carbon atoms, and is more preferably H;
R3 is H or an aliphatic hydrocarbon group, optionally substituted, having from 1 to 5, preferably from 1 to 2 carbon atoms, and is more preferably H;
R4 is H or an aliphatic hydrocarbon group, optionally substituted, having from 1 to 5, preferably from 1 to 2 carbon atoms, and is more preferably H;
R5 is H or an aliphatic hydrocarbon group, optionally substituted, having from 1 to 5, preferably from 1 to 2 carbon atoms, and is more preferably H;
m is an integer from 3 to 7,
said process comprising the following steps in sequence:
(A) reacting under addition conditions said compound of formula (I) with said compound of formula (II) in the presence of an organic or inorganic basic catalyst, in the absence or in the presence of a solvent, obtaining a compound of formula (III); 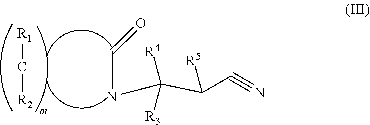
(B) subjecting said compound of formula (III) as obtained in step (A), preferably in the absence of intermediate purification steps of the compound from the other reaction products, to reduction by a reaction with hydrogen in the presence of a catalyst based on metals of groups 8, 9 and 10 of the periodic table, or noble metals, wherein said catalyst is not of the Raney type or of the sponge type, to obtain the corresponding amine of formula (IV) and optionally separate it from the reaction solvent; and 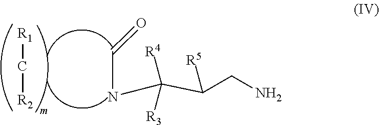
(C) subjecting said amine to dehydration in the presence of an acid catalyst, obtaining the corresponding amidine of formula (V).

2. The process according to claim 1, wherein said catalyst is selected from the group consisting of Iron, Cobalt, Nickel, Ruthenium, Rhodium, Palladium, Osmium, Iridium or Platinum based catalysts.

3. The process according to claim 1, wherein (I) is ε-Caprolactam, (II) is acrylonitrile, and (V) is 1,8-Diazabicyclo [5,4,0] undec-7-ene (DBU).

4. The process according to claim 1, wherein in step (A) the molar ratio (II)/(I) ranges from 1.4 to 0.7, or from 0.8 to 1.3, or about 1.1.

5. The process according to claim 1, wherein step (A) is carried out at temperatures from 20 to 140° C., or from 40 to 110° C., or from 60 to 80° C., and pressures from 0.1 to 6 barA, or from 0.1 to 4 barA, or at atmospheric pressure, for times that range, depending on the type of reagents (I) and (II), temperature and pressure, from 0.5 to 10 hours, or from 0.5 to 8 hours, or from 0.8 to 4 hours.

6. The process according to claim 1, wherein, when step (A) is carried out in the presence of a solvent, said solvent is selected from a polar solvent such as a linear, branched or cyclic ether, selected from methyl tert-butyl ether (MTBE) and tetrahydrofuran (THF), or an alcohol having from 1 to 6 carbon atoms selected from methanol, ethanol, isopropyl alcohol and tert-butyl alcohol or an aromatic solvent selected from benzene, toluene, xylenes and ethylbenzene, or an aliphatic hydrocarbon selected from heptane or cyclohexane; the amount of solvent being preferably from 5 to 70%, or from 5 to 50%, or from 15 to 40% by weight with respect to the total of the reaction mixture.

7. The process according to claim 1, wherein in step (A) the catalyst is selected from the group consisting of KOH, NaOH, LiOH, tetrabutylammonium hydroxide, DBU, primary amines, secondary amines, tertiary amines or other organic hydroxides.

8. The process according to claim 1, wherein in step (B) the intermediate of formula (III) coming from step (A) is subjected to the reduction reaction without being separated from the reaction mixture, except for a possible partial evaporation of the solvent, in the absence of ammonia and in the presence of water in a molar ratio H₂O/(III) from 0.01 to 1 or in a percentage from 0.1 to 11% by weight with respect to the reagent mixture.

9. The process according to claim 1, wherein the catalyst is a cobalt or nickel-based catalyst supported/bounded on a Lewis acid or a Lewis acid having Bronsted acid components, preferably selected from Al₂O₃ and SiO₂.

10. The process according to claim 1, wherein the reaction temperature in step (B) is from 30 to 250° C., or from 50 to 200° C., and the pressure is from 4 to 150 barA, or from 11 to 100 barA, or from 20 barA to 60 barA, in the absence of solvent or in the presence of an organic solvent selected from the group consisting of methanol, ethanol, isopropyl alcohol, tert-butyl alcohol, MTBE, THF, benzene, toluene, xylenes and ethylbenzene.

11. The process according to claim 1, wherein step (B) and step (C) are carried out continuously in a stirred reactor CSTR.

12. The process according to claim 1, wherein step (C) is carried out in the presence of para-toluenesulfonic acid, in the presence of a solvent preferably selected from xylenes and ethylbenzene, at a temperature from 90 to 270° C., or from 130 to 230° C., or from 150 to 200° C. and at a pressure from 0.08 to 5 barA, preferably from 0.5 to 3 barA, more preferably from 1 to 2 barA, continuously removing the water produced during dehydration.

13. The process according to claim 1, wherein step (C) is carried out in the presence of a catalyst selected from heterogeneous acid catalysts selected from Lewis acids, Lewis acids having Bronsted acid components, such as for example aluminium oxide (γ-Al₂O₃), silico alumina (SiO₂—Al₂O₃), acid earths selected from lanthanum oxide and zirconium oxide, or heterogeneous catalysts based on resins, sulfonated resins or ion exchange resins, said catalysts being optionally supported on inert carriers preferably selected from pumice, graphite and silica, in the absence of solvent or in the presence of a solvent preferably selected from xylenes and ethylbenzene, at a temperature from 90 to 270° C., or from 130 to 230° C., or from 150 to 200° C., and at a pressure from 0.08 to 5 barA, preferably from 0.5 to 3 barA, more preferably from 1 to 2 barA, continuously removing the water produced during dehydration.

14. The process according to claim 1, wherein steps (A), (B) and (C) are carried out continuously in the presence of a solvent selected from xylenes and ethylbenzene.

15. The process according to claim 1, wherein the reaction mixture leaving step (A) is fed to an evaporator or to a plate distillation column or with fillers to recover the solvent and possible reactants, and the liquid stream leaving the evaporator or the bottom of the distillation column, containing the addition products and the solubilized catalyst, is fed to a heat exchanger and is heated to a temperature from 30° C. to 250° C., or from 50° C. to 200° C., or from 100° C. to 160° C.; and wherein said stream coming from said exchanger is fed to a reactor for the reduction reaction according to step (B), at a pressure from 4 to 150 barA, or from 11 to 100 barA, or from 20 barA to 60 barA, said reactor being selected from a fixed bed reactor, or in a trickle bed arrangement, operating at a WHSV (Weight Hourly Space Velocity, relative to the sum of all the incoming currents) from 1 to 50, or from 3 to 10 h⁻.

16. A process for the synthesis of a compound of formula (IV): 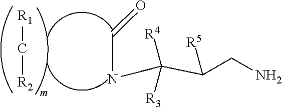
starting from a compound of formula (III): 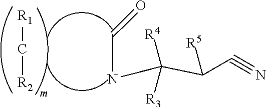
wherein:
R¹ is H or an aliphatic hydrocarbon group, optionally substituted, having from 1 to 5, preferably from 1 to 2 carbon atoms, and is more preferably H;
R2 is H or an aliphatic hydrocarbon group, optionally substituted, having from 1 to 5, preferably from 1 to 2 carbon atoms, and is more preferably H;
R3 is H or an aliphatic hydrocarbon group, optionally substituted, having from 1 to 5, preferably from 1 to 2 carbon atoms, and is more preferably H;
R4 is H or an aliphatic hydrocarbon group, optionally substituted, having from 1 to 5, preferably from 1 to 2 carbon atoms, and is more preferably H;
R5 is H or an aliphatic hydrocarbon group, optionally substituted, having from 1 to 5, preferably from 1 to 2 carbon atoms, and is more preferably H;
m is an integer from 3 to 7, more preferably from 3 to 6,
comprising a hydrogenation step of the compound of formula (III) by a reaction with hydrogen in the presence of a cobalt or nickel-based catalyst supported/bounded on a support selected from Al₂O₃ and SiO₂, wherein said catalyst is not of the Raney type or of the sponge type,
wherein the hydrogenation is carried out in the absence of ammonia and in the presence of water in a molar ratio H₂O/(III) from 0.01 to 1 or in a percentage from 0.1 to 11% by weight with respect to the reagent mixture, and wherein the reaction temperature is from 30 to 250° C., or from 50 to 200° C., and the pressure is from 4 to 150 barA, or from 11 to 100 barA, or from 20 barA to 60 barA, and wherein the hydrogenation is carried out in the absence of solvent or in the presence of an organic solvent selected from methanol, ethanol, isopropyl alcohol, tert-butyl alcohol, MTBE, THF, benzene, toluene, xylenes, ethylbenzene.
